(19) United States
(12) Patent Application Publication
(10) Pub. No.: US 2023/0010712 A1
(43) Pub. Date: Jan. 12, 2023

(54) METHODS, SYSTEMS, AND COMPOSITIONS FOR ACHIEVING A HEALTHY INTRAOCULAR PRESSURE FOLLOWING COMBINED GLAUCOMA FILTRATION SURGERY AND CATARACT EXTRACTION

(71) Applicant: RADIANCE THERAPEUTICS, INC., Tucson, AZ (US)

(72) Inventor: Laurence J. Marsteller, Tucson, AZ (US)

(21) Appl. No.: 17/782,940

(22) PCT Filed: Dec. 4, 2020
(86) PCT No.: PCT/US2020/063435

§ 371 Date: Jun. 6, 2022

Related U.S. Application Data
(60) Provisional application No. 62/944,952, filed on Dec. 6, 2019.

(51) Int. Cl. A61F 9/007

(52) U.S. Cl. CPC A61F 9/00781; A61F 9/0079

(57) ABSTRACT

Methods and systems for applying beta radiation to a treatment area, such as a target area of a bleb, in combination with combined glaucoma and cataract surgery. The methods and systems herein may help achieve and/or maintain a healthy intraocular pressure, maintain functioning blebs and/or drainage holes arising from glaucoma drainage procedures or surgeries, help avoid scar formation or wound reversion, inhibit or reduce fibrogenesis and/or inflammation in the blebs or surrounding areas, etc.

### CROSS-REFERENCES TO RELATED APPLICATIONS

This application claims the benefit of U.S. Provisional Application No. 62/944,952, filed on Dec. 6, 2019, the contents of which are incorporated by reference.

### FIELD OF THE INVENTION

The present invention relates to methods, systems, and compositions for achieving a healthy intraocular pressure following combined glaucoma and cataract surgery. For example, the present invention features methods and systems for treating glaucoma treatment-associated drainage blebs and/or holes, such as those associated with foreign bodies or other glaucoma procedures, with beta radiation to maintain functioning blebs and/or holes.

### BACKGROUND OF THE INVENTION

### Glaucoma

Glaucoma is the leading cause of irreversible blindness and represents a family of diseases with a characteristic optic neuropathy. Therapy for this group of diseases is principally focused at reducing the intraocular pressure (IOP) of the fluid inside the eye (aqueous humor), thus averting ongoing damage to the optic nerve.

Glaucoma is managed by attempting to lower the intraocular pressure (IOP). In the USA, Europe, and some other industrialized countries, the first line therapy is typically medication delivered by eye drops. Such medications include beta-blockers, prostaglandins, alpha-adrenergic agonists, and carbonic anhydrase inhibitors. For patients who fail medication and in other parts of the world where there are economic and distribution barriers to the practicality of daily medication and frequent follow up, the treatment regime is primarily surgical interventions.

One way to prevent vision loss from glaucoma is to lower intraocular pressure with drainage surgery that shunts fluid out of the eye through a channel created during a trabeculectomy procedure, by implanting a flow-controlled drainage device during Minimally Invasive Glaucoma Surgery (MIGS), or by the use of other surgical procedures such as Minimally Invasive Micro Sclerostomy (MIMS), trabeculectomy, or other devices. These systems and procedures allow drainage of the aqueous humor from within the eye to a small reservoir (termed a “bleb”) under the conjunctiva, from where the aqueous humor is later reabsorbed.

With current glaucoma treatments (e.g., MIMS, MIGS, trabeculectomy, etc.), scar tissue often compromises the bleb or other surrounding structures (e.g., drainage channels associated with MIMS), ultimately impeding or blocking the flow of excess fluid. Despite compelling therapeutic advantages over nonsurgical treatments, drainage surgery and devices are clinically limited by postoperative scarring.

Attempts to address this include the application of antimetabolites such as mitomycin C (MMC) and 5-fluorouracil (5FU). These antimetabolites are used in liquid form and are delivered either by injection or by placing microsurgical sponges soaked in the drug directly onto the operative site underneath the conjunctiva. One of the problems associated with antimetabolites (e.g., MMC and 5FU) is that they do not preserve blebs well. By some reports, the failure rate by three years approaches 50%.

An additional problem with glaucoma treatments is the frequent co-existence and exacerbation of cataracts. Cataract extraction surgery, such as phoacoemulsification can be performed in combination a with glaucoma treatment. However, there is consensus in the glaucoma community that combining any form of external glaucoma drainage surgery with cataract extraction results in a poorer outcome. As an example, the use of a PRESERFLO™ MicroShunt (Santen, previously InnFocus MicroShunt®) combined with cataract surgery is a convenient method of combining cataract surgery with a drainage procedure. However, without adequate control of the wound healing response, it is likely that the intraocular pressure lowering would be sub-optimal.

As another example, the use of a Xen Gel Stent (Allergan) combined with cataract surgery may be a convenient method of combining cataract surgery with a drainage procedure. However, without adequate control of the wound healing response, it is likely that the intraocular pressure lowering would be sub-optimal.

It has been surprisingly discovered that the use of beta radiation to maintain a functional drainage bleb and help lower IOP can reduce conjunctival inflammation associated with glaucoma surgery (e.g., the implantation of a MIGS implant or foreign body) to such a degree that beta radiation is still effective even with the inclusion of cataract surgery.

In a randomized controlled trial using beta radiation or 5 Fluorouracil (5FU) as an adjunct in combined phacotrabeculectomy surgery, patients received either 1000 cGy beta radiation via an 8 mm disc applicator with Sr-90/Y-90 or 5FU as a soaked cotton pledget under the bleb or via injection (Dhalla et al., 2016, PLoS ONE 11(9): e0161674). Patients were followed for 12 months post-surgery. Surgical success was judged by three criteria: IOP≤16 mmHg (and not on ocular hypotensive treatment), ≤21 mmHg, and a reduction of 30% or more in IOP, Per Dhalla et al., “If treatment success is defined as a reduction of 30% or more in IOP and an IOP≤21 mmHg, then at one year the proportion of cross-sectional successful outcomes in the 5FU and beta radiation arms were 82.6% and 82.7%, respectively (P=0.99).” Without wishing to limit the present invention to any theory or mechanism, given Dhalla's finding that the use of beta radiation did not prove to be more effective than 5FU when used as an adjunct in combined phacotrabeculectomy surgery, it is believed that one of ordinary skill in the art would not expect that beta radiation would yield more positive outcomes when used as an adjunct to cataract surgery and glaucoma surgery involving the introduction of a foreign body into the eye.

Without wishing to limit the present invention to any theory or mechanism, it is believed that it has not yet been possible to achieve an intraocular pressure around 10 mmHg after combined glaucoma filtration surgery and cataract extraction, even when antimetabolites such as mitomycin-C are used.

### SUMMARY OF THE INVENTION

The present invention features methods and systems for applying radiation to a treatment area, such as a target area of a bleb, in combination with combined glaucoma and cataract surgery. The methods and systems herein may be used to apply beta radiation to a target area in the eye to help maintain functioning blebs and/or drainage holes arising from glaucoma drainage procedures or surgeries, to help avoid scar formation or wound reversion, to inhibit or reduce fibrogenesis and/or inflammation in the blebs or surrounding areas, etc. The present invention is not limited to the applications disclosed herein.

As used herein, the term “treatment area” or “target area” may refer to the tissue that is desired or expected to be treated with beta radiation. The treatment area or target area may be defined as a particular plane of a certain size and a particular depth within an area of tissue being exposed to the beta radiation.

The methods feature applying a therapeutic dose of beta radiation to the target site (e.g., drainage site or other appropriate site) at or around the time of combined cataract surgery and glaucoma surgery (e.g., implantation of a drainage device, e.g., MIGS implantation), e.g., before glaucoma surgery, after glaucoma surgery, before cataract surgery, after cataract surgery, etc.

The methods and systems herein help provide an optimized dose distribution across the target area or treatment area. Without wishing to limit the present invention to any theory or mechanism, as used herein, the term “optimized dose distribution” may refer to a dose across a particular plane of a certain size at a particular depth on or within the target area or treatment area that is substantially uniform and therapeutic in dose. For example, the dose across the particular plane on or within the target may vary by no more than a certain percentage of the maximum dose.

FIG. 2 illustrates a non-limiting example of a plane of a target area. The size and dimensions (and depth) of the target and target plane may vary. In some embodiments, the diameter of the target area is 6 mm. In some embodiments, the diameter of the target area is 7 mm, In some embodiments, the diameter of the target area is 8 mm. In some embodiments, the diameter of the target area is 9 mm. In some embodiments, the diameter of the target area is 10 mm. In some embodiments, the diameter of the target area is 11 mm. In some embodiments, the depth of the target area, e.g., the depth of a plane of the target area, is 0 mm (e.g., in contact with a brachytherapy deliver system, e.g., a radionuclide brachytherapy system). In some embodiments, the depth of the target area, e.g., the depth of a plane of the target area, is 0.1 mm. In some embodiments, the depth of the target area, e.g., the depth of a plane of the target area, is 0.2 mm. In some embodiments, the depth of the target area, e.g., the depth of a plane of the target area, is 0.3 mm. In some embodiments, the depth of the target area, e.g., the depth of a plane of the target area, is 0.4 mm. In some embodiments, the depth of the target area, e.g., the depth of a plane of the target area, is 0.5 mm. In some embodiments, the depth of the target area, e.g. the depth of a plane of the target area, is 0.6 mm, In some embodiments, the depth of the target area is from 0 to 0.4 mm.

Alternatively, “optimized dose distribution” may also mean that the dose distribution is varied across the lesion in a specific pattern with the intention to best affect the therapeutic outcome. In one example, the dose distribution across the diameter/plane at the treatment depth varies such that the areas at the edges of the bleb receive a higher dose relative to the center. In one example, the dose distribution across the diameter/plane at the treatment depth varies such that the area at the MIGS device outflow orifice receives a boosted dose compared to other areas, In one example, the dose distribution across the diameter/plane at the treatment depth varies such that the edges of the bleb and also the area at the MIGS device outflow orifice both receive a boosted dose, In one example, the dose is attenuated over a specified area, In one example, the dose is attenuated over the cornea.

Beta radiation attenuates quickly with depth. In some embodiments, the term “optimized dose distribution” includes an appropriate dose through the depth of the target tissue, The clinical dosage depth may be determined by the thickness of the conjunctiva and associated tenon's capsule of a functional bleb. As a non-limiting example, for MIGS surgery, the focus area may be approximately 3 mm above the superior limbus. Howlet et al., found the mean thickness of the conjunctival and Tenon's layer to be 393±67 microns ranging from 194 to 573 microns using optical coherence tomography (OCT) in glaucoma patients (Howlet J et al., Journal of Current Glaucoma Practice 2014, 8(s):63-66). In an earlier study, Zhang et al. found conjunctival thickness to be 238±51 microns in healthy individuals using OCT analysis and concluded OCT accurately measures the cross-sectional structures of conjunctival tissue with high resolution (Zhang et al., Investigative Ophthalmology & Visual Science 2011, 52(10):7787-7791). Based on the Howlet study, the target tissue thickness may range from 150 to 700 microns, or from 10 to 700 microns, etc, In one example, the dose distribution from the surface through the depth of the target tissue allows for a therapeutic dose within the tissue to the limits of the rapidly attenuating beta rays.

The present invention features a radioisotope that emits beta radiation for use in a method of treating both glaucoma and cataracts.

In some embodiments, the method comprises performing a glaucoma drainage surgery on an eye of a patient that forms a bleb in a subconjunctival space or between the conjunctiva and Tenon's capsule and the glaucoma drainage surgery allows aqueous humor to drain into the bleb (e.g., MIGS, MIMS, trabeculectomy, etc.); performing cataract surgery; and applying a therapeutic dose of the beta radiation from the radioisotope to a target area of the eye, wherein the target area is associated with the bleb, a glaucoma drainage implant, or a drainage channel, or a combination thereof, etc. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

In some embodiments, the method comprises performing a glaucoma drainage surgery on an eye of a patient wherein an implant (e.g., MIGS implant) is implanted trans-sclerally to form a bleb in a subconjunctival space or between the conjunctiva and Tenon's capsule, the glaucoma drainage surgery allows aqueous humor to drain into the bleb; performing cataract surgery; and applying a therapeutic dose of the beta radiation from the radioisotope to a target area of the eye, wherein the target area is associated with the bleb, the implant, or both the bleb and implant.

The present invention also features a radioisotope that emits beta radiation for use for use in preventing or reducing scar formation in a drainage bleb or drainage channel in an eye being treated or having been treated with glaucoma surgery (e.g., MIGS, MIMS, trabeculectomy, etc.) and cataract surgery, characterized in that the radioisotope is administered to the eye such that a therapeutic dose of beta radiation from the radioisotope is applied to a target area of the eye. The target area may be associated with the drainage bleb, a drainage channel, or a glaucoma drainage implant, or a combination thereof. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a radioisotope that emits beta radiation for use for use in preventing or reducing scar formation in a drainage bleb in an eye being treated or having been treated with (i) glaucoma drainage surgery wherein an implant (e.g., MIGS implant) is implanted trans-sclerally to form a bleb in a subconjunctival space or between the conjunctiva and Tenon's capsule and aqueous humor can drain into the drainage bleb, and (ii) cataract surgery, characterized in that the radioisotope is administered to the eye such that a therapeutic dose of beta radiation from the radioisotope is applied to a target area of the eye. The target area may be associated with the drainage bleb, the implant, or both the bleb and implant.

The present invention also features a radioisotope that emits beta radiation for use for use in a method for reducing intraocular pressure (IOP) in an eye being treated or having been treated with glaucoma surgery (e.g., MIGS, MIMS, trabeculectomy, etc.) and cataract surgery, characterized in that the radioisotope is administered to the eye such that a therapeutic dose of beta radiation from the radioisotope is applied to a target area of the eye. The target area may be associated with the drainage bleb, a drainage channel, or a glaucoma drainage implant, or a combination thereof. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a radioisotope that emits beta radiation for use for use in a method for reducing intraocular pressure (IOP) in an eye being treated or having been treated with (i) glaucoma drainage surgery wherein an implant (e.g., MIGS implant) is implanted trans-sclerally to form a bleb in a subconjunctival space or between the conjunctiva and Tenon's capsule and aqueous humor can drain into the drainage bleb, and (ii) cataract surgery, characterized in that the radioisotope is administered to the eye such that a therapeutic dose of beta radiation from the radioisotope is applied to a target area of the eye. The target area may be associated with the drainage bleb, the implant, or both the bleb and implant.

The present invention also features a composition comprising a source of beta radiation for use in a method for achieving a healthy intraocular pressure (IOP) in a human eye being treated or having been treated for glaucoma (e.g., MIGS, MIMS, trabeculectomy) and cataracts, characterized in that the composition is administered to the eye such that beta radiation from the source of beta radiation is applied to a target area of the eye. The target area may be associated with the drainage bleb, a drainage channel, or a glaucoma drainage implant, or a combination thereof. In some embodiments, the radioisotope comprises Strontium-90 (Sr-90), Phosphorus-32 (P-32), Ruthenium 106 (Ru-106), Yttrium 90 (Y-90), or a combination thereof. In some embodiments, the therapeutic dose is from 500-1000 cGy. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a composition comprising a source of beta radiation for use in a method for achieving a healthy intraocular pressure (IOP) in an eye being treated or having been treated with (i) glaucoma drainage surgery (e.g., MIGS) wherein an implant (e.g., MIGS implant) is implanted trans-sclerally to form a bleb in a subconjunctival space or between the conjunctiva and Tenon's capsule and aqueous humor can drain into the drainage bleb, and (ii) cataract surgery, characterized in that the composition is administered to the eye such that beta radiation from a source of beta radiation is applied to a target area of the eye. The target area may be associated with the drainage bleb, the implant, or both the bleb and implant.

The present invention also features a method of reducing intraocular pressure (IOP) of an eye being treated or having been treated with both glaucoma drainage surgery (e.g., MIGS, MIMS, trabeculectomy, etc.) and cataract surgery, wherein the glaucoma surgery allows aqueous humor to drain into a bleb in a subconjunctival space or space between a conjunctiva and Tenon's capsule. In some embodiments, the method comprises applying a therapeutic amount of beta radiation from a radioisotope to a target area of the eye, wherein the target area is associated with the bleb, a glaucoma drainage implant, or a drainage channel, or a combination thereof; wherein the therapeutic amount of beta radiation helps maintain a functioning drainage bleb. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a method of reducing intraocular pressure (IOP) in an eye being treated or having been treated with (i) glaucoma drainage surgery (e.g., MIGS) wherein an implant (e.g., MIGS implant) is implanted trans-sclerally to form a bleb in a subconjunctival space or between the conjunctiva and Tenon's capsule and aqueous humor can drain into the drainage bleb, and cataract surgery, said method comprising applying a therapeutic amount of beta radiation from a radioisotope to a target area of the eye, wherein the target area is associated with the bleb, the implant, or both the bleb and implant; wherein the therapeutic amount of beta radiation helps maintain a functioning drainage bleb.

The present invention also features a method of reducing conjunctival inflammation in an eye being treated or having been treated with both glaucoma drainage surgery (e.g., MIGS, MIMS, trabeculectomy, etc.) and cataract surgery, wherein the glaucoma surgery allows aqueous humor to drain into a drainage bleb in a subconjunctival space or space between a conjunctiva and Tenon's capsule; In some embodiments, the method comprises applying a therapeutic amount of beta radiation from a radioisotope to a target area of the eye, wherein the target area is associated with the bleb, a glaucoma drainage implant, or a drainage channel, or a combination thereof; wherein the beta radiation causes cell cycle arrest in fibroblasts on the Tenon's capsule to inhibit or reduce the fibrotic process and conjunctival inflammation. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a method of reducing conjunctival inflammation in an eye being treated or having been treated with (i) glaucoma drainage surgery (e.g., MIGS) wherein an implant (e.g., MIGS implant) is implanted trans-sclerally to form a bleb in a subconjunctival space or between the conjunctiva and Tenon's capsule and aqueous humor can drain into the drainage bleb, and (ii) cataract surgery, said method comprising applying a therapeutic amount of the beta radiation from the radioisotope to a target area of the eye, wherein the target area is associated with the bleb, the implant, or both the bleb and implant; wherein the beta radiation causes cell cycle arrest in fibroblasts on the Tenon's capsule to inhibit or reduce the fibrotic process and conjunctival inflammation.

The present invention also features a method of achieving a healthy intraocular pressure (IOP) in an eye being treated or having been treated with both glaucoma drainage surgery (e.g., MIGS, MIMS, trabeculectomy) and cataract surgery, wherein the glaucoma surgery allows aqueous humor to drain into a bleb in a subconjunctival space or space between a conjunctiva and Tenon's capsule. In some embodiments, the method comprises applying a therapeutic amount of beta radiation from a radioisotope to a target area of the eye, wherein the target area is associated with the bleb, a glaucoma drainage implant, or a drainage channel, or a combination thereof; wherein the therapeutic amount of beta radiation helps maintain a functioning drainage bleb so as to achieve a healthy IOP. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a method of achieving a healthy intraocular pressure (IOP) in an eye being treated or having been treated with (i) glaucoma drainage surgery (e.g., MIGS) wherein an implant (e.g., MIGS implant) is implanted trans-sclerally to form a bleb in a subconjunctival space or between the conjunctiva and Tenon's capsule and aqueous humor can drain into the drainage bleb, and (ii) cataract surgery, said method comprising applying a therapeutic amount of beta radiation from a radioisotope to a target area of the eye, wherein the target area is associated with the bleb, the implant, or both the bleb and implant; wherein the therapeutic amount of beta radiation helps maintain a functioning drainage bleb so as to achieve a healthy IOP,

The present invention also features a method of treating glaucoma and cataracts. In some embodiments, the method comprises performing a glaucoma drainage surgery (e.g., MIGS, MIMS, trabeculectomy, etc.), wherein the glaucoma drainage surgery allows aqueous humor to drain into a bleb in a subconjunctival space or space between a conjunctiva and Tenon's capsule; performing cataract surgery; and applying a therapeutic dose of the beta radiation from the radioisotope to a target area of the eye, wherein the target area is associated with the bleb, a glaucoma drainage implant, or a drainage channel, or a combination thereof. The method may be effective for reducing intraocular pressure (IOP). The method may be effective for achieving a healthy intraocular pressure (IOP). In some embodiments, the glaucoma surgery is Minimally. Invasive Glaucoma Surgery (MIGS). In some embodiments, the glaucoma surgery is Minimally Invasive Micro Sclerostomy (MIMS). In some embodiments, the glaucoma surgery is trabeculectomy.

The present invention also features a method of treating glaucoma and cataracts, said method comprising: performing a glaucoma drainage surgery (e.g., MIGS) in an eye, wherein an implant (e.g., MIGS implant) is implanted trans-sclerally to form a bleb in a subconjunctival space or between the conjunctiva and Tenon's capsule and aqueous humor can drain into the drainage bleb; performing cataract surgery on the eye; and applying a therapeutic dose of the beta radiation from a radioisotope to a target area of the eye, wherein the target area is associated with the bleb, the implant, or both the bleb and implant. The method may be effective for reducing intraocular pressure (IOP). The method may be effective for achieving a healthy intraocular pressure (IOP).

The methods herein may be effective for lowering intraocular pressure (IOP). In some embodiments, the method is effective for maintaining a functioning drainage bleb. In some embodiments, the method is effective for inhibiting or reducing fibrogenesis and inflammation in the bleb, around the drainage implant, or around the drainage channel. In some embodiments, the method is effective for reducing conjunctival inflammation.

Referring to any of the embodiments herein, the radioisotope may comprise Strontium-90 (Sr-90), Phosphorus-32 (P-32), Ruthenium 106 (Ru-106), Yttrium 90 (Y-90), or a combination thereof. In some embodiments, the therapeutic dose is from 500-1000 cGy. In some embodiments, the therapeutic dose is from 450-1050 cGy.

Referring to any of the embodiments herein, in some embodiments, the method further comprises administering a drug to the target area. In some embodiments, the drug is mitomycin C or 5 fluorouracil. In some embodiments, the drug is an anti-VEGF composition.

Referring to any of the embodiments herein, in some embodiments, beta radiation is applied to the target after performing the glaucoma drainage surgery. In some embodiments, beta radiation is applied to the target before performing the glaucoma drainage surgery. In some embodiments, beta radiation is applied to the target while performing the glaucoma drainage surgery. In some embodiments, beta radiation is applied to the target before and after performing the glaucoma drainage surgery.

Referring to any of the embodiments herein, in some embodiments, IOP is reduced to 12 mmHg or less. In some embodiments, IOP is reduced to 10 mmHg or less. In some embodiments, IOP is reduced to from 5 to 10 mmHg. In some embodiments, IOP is reduced to from 5 to 12 mmHg. In some embodiments, IOP is reduced to from 8 to 10 mmHg. In some embodiments, IOP is reduced to from 8 to 12 mmHg.

Referring to any of the embodiments herein, the method may be effective for reducing IOP by a certain amount for a certain length of time after treatment. In some embodiments, the method is effective for reducing IOP by 20% or more 6 months after treatment. In some embodiments, the method is effective for reducing IOP by 30% or more 6 months after treatment. In some embodiments, the method is effective for reducing IOP by 40% or more 6 months after treatment. In some embodiments, the method is effective for reducing IOP by 50% or more 6 months after treatment. In some embodiments, the method is effective for reducing IOP by 20% or more 12 months after treatment. In some embodiments, the method is effective for reducing IOP by 30% or more 12 months after treatment. In some embodiments, the method is effective for reducing IOP by 40% or more 12 months after treatment. In some embodiments, the method is effective for reducing IOP by 50% or more 12 months after treatment. In some embodiments, the method is effective for reducing IOP by 20% or more 24 months after treatment. In some embodiments, the method is effective for reducing IOP by 30% or more 24 months after treatment. In some embodiments, the method is effective for reducing IOP by 40% or more 24 months after treatment. In some embodiments, the method is effective for reducing IOP by 50% or more 24 months after treatment. In some embodiments, the method is effective for reducing IOP by 20% or more 36 months after treatment. In some embodiments, the method is effective for reducing IOP by 30% or more 36 months after treatment. In some embodiments, the method is effective for reducing IOP by 40% or more 36 months after treatment. In some embodiments, the method is effective for reducing IOP by 50% or more 36 months after treatment.

Referring to any of the embodiments herein, in some embodiments, the method is effective for reduction of IOP and subsequent stabilization of IOP, e.g., IOP is stabilized for a certain length of time. In some embodiments, stabilization of IOP is wherein the IOP does not increase by more than 10% at 3 months after treatment. In some embodiments, stabilization of IOP is wherein the IOP does not increase by more than 10% at 6 months after treatment. In some embodiments, stabilization of IOP is wherein the IOP does not increase by more than 10% at 12 months after treatment. In some embodiments, stabilization of IOP is wherein the IOP does not increase by more than 10% at 24 months after treatment. In some embodiments, stabilization of IOP is wherein the IOP does not increase by more than 10% at 36 months after treatment. In some embodiments, stabilization of IOP is wherein the IOP does not increase by more than 20% at 3 months after treatment. In some embodiments, stabilization of IOP is wherein the IOP does not increase by more than 20% at 6 months after treatment. In some embodiments, stabilization of IOP is wherein the IOP does not increase by more than 20% at 12 months after treatment. In some embodiments, stabilization of IOP is wherein the IOP does not increase by more than 20% at 24 months after treatment. In some embodiments, stabilization of IOP is wherein the IOP does not increase by more than 20% at 36 months after treatment. In some embodiments, stabilization of IOP is wherein the IOP does not increase by more than 25% at 24 months after treatment. In some embodiments, stabilization of IOP is wherein the IOP does not increase by more than 25% at 36 months after treatment.

In some embodiments, the glaucoma surgery is Minimally Invasive Glaucoma Surgery (MICS). In some embodiments, the glaucoma surgery is Minimally Invasive Micro Sclerostomy (MIMS). In some embodiments, the glaucoma surgery is trabeculectomy.

In some embodiments, inhibiting or reducing fibrogenesis and inflammation in the bleb is measured according to a predetermined bleb grading scale. The predetermined bleb grading scale is the Moorfields bleb grading scale (MBGS) and/or the Indiana Bleb Appearance Grading Scale (IBAGS).

In some embodiments, the beta radiation is applied to the target using an applicator.

In some embodiments, the target is at least a portion of a bleb. In some embodiments, the target comprises an entire bleb. In some embodiments, the target area surrounds an end of a Minimally Invasive Glaucoma Surgery (MIGS) implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel. In some embodiments, the target comprises a portion of a bleb. In some embodiments, the target area comprises an end of a Minimally Invasive Glaucoma Surgery (MIGS) implant.

Referring to any of the embodiments herein, in some embodiments, the method is effective for preventing further loss of vision for a certain time period. Loss of vision may be determined using techniques, measurements, and scales well known to one of ordinary skill in the art. In some embodiments, the method prevents further loss of vision for at least 2 months after treatment. In some embodiments, the method prevents further loss of vision for at least 3 months after treatment. In some embodiments, the method prevents further loss of vision for at least 4 months after treatment. In some embodiments, the method prevents further loss of vision for at least 5 months after treatment. In some embodiments, the method prevents further loss of vision for at least 6 months after treatment. In some embodiments, the method prevents further loss of vision for at least 7 months after treatment. In some embodiments, the method prevents further loss of vision for at least 8 months after treatment. In some embodiments, the method prevents further loss of vision for at least 9 months after treatment. In some embodiments, the method prevents further loss of vision for at least 12 months after treatment. In some embodiments, the method prevents further loss of vision for at least 18 months after treatment. In some embodiments, the method prevents further loss of vision for at least 24 months after treatment.

As previously discussed, the present invention provides therapeutic doses of beta radiation. As shown in FIG. 1 the present invention provides a relatively flat and consistent dose across a large portion of the target area.

The present invention also features a radionuclide brachytherapy source (RBS) system that emits beta radiation for use in a method of treating both glaucoma and cataracts (e.g., for helping to lower IOP). In some embodiments, the method comprises performing a glaucoma drainage surgery on an eye of a patient to form a bleb in a subconjunctival space or between the conjunctiva and Tenon's capsule, and to allow aqueous humor to drain into the bleb; performing cataract surgery; and applying a therapeutic dose of beta radiation from the RBS system to a target area associated with the bleb, a drainage channel, a drainage implant, or a combination thereof. In some embodiments, the glaucoma drainage surgery is MIGS, MIMS, or trabeculectomy.

The present invention also features a method of reducing intraocular pressure (IOP) in an eye being treated or having been treated with (i) glaucoma drainage surgery to form a bleb in a subconjunctival space or between the conjunctiva and Tenon's capsule and to allow aqueous humor to drain into the drainage bleb, and (ii) cataract surgery. In some embodiments, the method comprises applying a therapeutic amount of beta radiation from a radionuclide brachytherapy (RBS) system to a target area associated with the bleb, a drainage channel, a drainage implant, or a combination thereof. In some embodiments, the glaucoma drainage surgery is MIGS, MIMS, or trabeculectomy.

In some embodiments, the method is effective for lowering intraocular pressure (IOP). In some embodiments, the therapeutic amount of beta radiation helps maintain a functioning drainage bleb. In some embodiments, the therapeutic amount of beta radiation helps reduce conjunctival inflammation.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a comparison of a dose profile from a legacy device (prior art) and a dose profile of a system of the present invention.

FIG. 2 shows an example of a target plane within a target area, relative to a radionuclide brachytherapy system (RBS system).

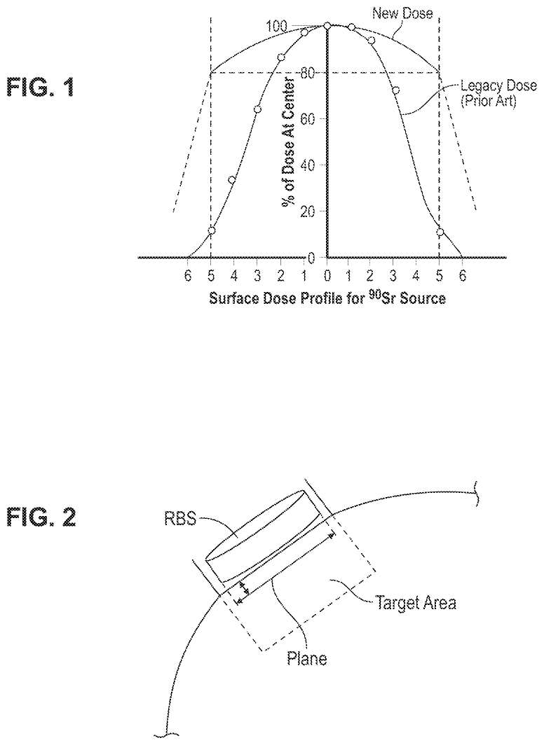

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references.

Dosimetry techniques include film dosimetry. In one example the RBS is applied to radiographic film, for example Gafchromic™ film. The dose at various depths can also be measured by placing an intervening material, such as Plastic Water™, of known thicknesses between the RBS and the film. A transmission densitometer in conjunction with a film optical density vs. dose chart, allows for the film opacity to be measured and then converted to delivered dose. Other methods include Thermoluminescent methods (TLD chips). TLD chips are small plastic chips with millimeter dimensions having a crystal lattice that absorbs ionizing radiation.

Dose variation is described as that across the diameter assuming a central point maximum dose. However, in practice it has been demonstrated that the maximum dose may be off center. Thus, a description of variation of dose across the diameter may also include the variation of dose over the area, and though the depth.

In general use in the profession of ophthalmology the term “conjunctivae” may refer to the conjunctivae in combination with the Tenon's capsule. Also, in general use in the profession of ophthalmology the term “conjunctivae” may refer to the conjunctivae alone, not including the Tenon's capsule. References herein to “conjunctivae” can include either and/or both meanings.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Beam Modification: Desirable modification in the spatial distribution of radiation (e.g., within the patient) by insertion of any material in the beam path. Beam modification increases conformity allowing a higher dose delivery to the target, while sparing more of normal tissue simultaneously. There are four main types of beam modification: (1) Shielding: To eliminate radiation dose to some special parts of the zone at which the beam is directed. In general use is the fabrication of low-melting-temperature alloy (Lipowitz metal or Cerroblend) shielding blocks that are custom made for the individual patient and used to shield normal tissue and critical organs. For example, during total body irradiation (TBI), customized shielding blocks are positioned in front of the lungs to reduce radiation dose. (2) Compensation: To allow normal dose distribution data to be applied to the treated zone, when the beam enters obliquely through the body, or where different types of tissues are present. (3) Wedge filtration: Where a special tilt in isodose curves is obtained. (4) Flattening: Where the spatial distribution of the natural beam is altered by reducing the central exposure rate relative to the peripheral. In general use is a beam flattening filter that reduces the central exposure rate relative to that near the edge of the beam. This technique is used for linear accelerators. The filter is designed so that the thickest part is in the center. These are often constructed of copper or brass.

Innovations such as stereotaxic radiotherapy, intensity modulated radiation therapy, and conformal radiotherapy are also applied towards the goal of sparing normal tissue and critical organs. For example, Linear Accelerators designed with Multileaf Collimators have, in many circumstances, replaced shielding books.

Brachytherapy (see also Radionuclide Brachytherapy Source (RBS): According to the American Association of Physicists in Medicine (AAPM), brachytherapy is “the clinical use of small encapsulated radioactive sources at a short distance from the target volume for irradiation of malignant tumors or nonmalignant lesions,” Generally, in medical practice, brachytherapy can be categorized as topical or plaque brachytherapy, intracavitary, and interstitial.

Some implementations of brachytherapy employ permanently implanted Radionuclide Brachytherapy Sources (RBSs). For example, in Low Dose Rate (LDR) brachytherapy for prostate cancer, a standard of care treatment, radioactive Iodine-125 RBSs are placed directly into the prostate where they remain indefinitely. In another implementation, High Dose Rate (HDR) brachytherapy TheraSpheres are infused into the arteries that feed liver tumors. These microspheres then embolize, lodging themselves in the liver's capillaries and bathing the malignancy in high levels of yttrium-90 radiation. In both these implementations, the total dose is given by consuming the entire radioisotope. Some other implementations of brachytherapy employ a transient placement of the RBS. For example, in after-loaded High Dose Rate (HDR) brachytherapy, very tiny plastic catheters are placed into the prostate gland, and a series of radiation treatments is given through these catheters. A computer-controlled machine pushes a single highly radioactive iridium-192 RBS into the catheters one by one for a specified dwell time at locations throughout the volume being irradiated. The catheters are then easily pulled out, and no radioactive material is left at the prostate gland. Another example of transient placement of an RBS includes prophylactic therapy for restenosis of coronary arteries after stent implantation. This is a non-malignant condition that has been successfully treated by placing a catheter into the coronary artery, then inserting an HDR radioactive source into the catheter and holding it there for a predetermined time in order to deliver a sufficient dose to the vessel wall.

Drainage Device or Drainage System: Any or a combination of the general and specific approaches for draining aqueous humor, such as the therapeutic and devices described herein, e.g., minimally invasive glaucoma surgery (MIGS) devices and surgery, Minimally Invasive Micro Sclerostomy (MIMS) devices and surgery, trabeculectomy surgery, sclerostomy, etc., that are employed to reduce intraocular pressure (IOP) by means of surgical intervention with or without a device.

Flow Controlled Stents (see also Minimally Invasive Glaucoma Surgery (MIGS)): Some MIGS-associated devices control flow of the aqueous humor. For example, the XEN® gel stent (Allergan) is a gelatin and glutaraldehyde tube, which is preloaded in a disposable injector and implanted using an ab interna approach. The surgeon inserts the injector through a dear cornea incision and tunnels through the sclera at or anterior to Schlemm's canal to deploy the distal portion of the stent within the subconjunctival space. This creates a pathway for aqueous to flow from the anterior chamber to the subconjunctival space, forming a bleb. Another flow-controlled stent is the InnFocus MicroShunt® (InnFocus, Santen). The surgeon inserts this device into the anterior chamber through an ab externo approach, creating a bleb in the subconjunctival space.

Functioning Drainage Bleb: A bleb that is effective for draining aqueous humor from the eye to reduce intraocular pressure (IOP) of the eye to an appropriate level.

Early bleb grading systems included those proposed by Kronfeld (1969), Migdal and Hitchings (1983), and Picht and Grehn (1998). Subsequent bleb grading systems identified and incorporated a graded assessment of various bleb parameters such as vascularity, height, width, microcystic changes, encystment and diffuse/demarcated zones.

There are two recently described grading systems for clinical grading of filtering surgery blebs: the Moorfields Bleb Grading System (MBGS) and the Indiana Bleb Appearance Grading Scale (IBAGS), The MBGS built upon the system used for this tele-medicine study and expanded it to include an assessment of vascularity away from the center of the bleb and a way to represent mixed-morphology blebs. In this scheme, central area (1-5), maximal area (1-5), bleb height (1-4) and subconjunctival blood (0-1) were assessed. In addition, three areas of the bleb were graded separately for vascularity, including bleb center conjunctiva, peripheral conjunctiva and non-bleb conjunctiva. Vascularity in each area was assigned a score from 1 to 5. A study found good inter-observer agreement and clinical reproducibility in the IBAGS and MBGS (Wells A P, Ashraff N N, Hall R C, et al. Comparison of two clinical bleb grading systems. Ophthalmology 2006;113:77-83.)

The Moorfields bleb grading system was developed as the importance of bleb appearance to outcome was realized. Blebs that develop thin avascular zones are at increased risk of leakage and late hypotony as well as sight threatening bleb related infections.

The Indiana Bleb Appearance Grading Scale is a system for classifying the morphologic slit lamp appearance of filtration blebs. The Indiana Bleb Appearance

Grading Scale contains a set of photographic standards illustrating a range of filtering bleb morphology selected from the slide library of the Glaucoma Service at the Indiana University Department of Ophthalmology, These standards consist of slit lamp images for grading bleb height, extent, vascularity, and leakage with the Seidel test. For grading, the morphologic appearance of the filtration bleb is assessed relative to the standard images for the 4 parameters and scored accordingly.

For reference, a failed or failing bleb may have “restricted posterior flow with the so-called ‘ring of steel’,” e.g., a ring of scar tissue or fibrosis adhering the conjunctiva to the sclera at the periphery of the bleb that restricts the flow of aqueous humor (see Dhingra S, Khaw P T. The Moorfields Safer Surgery System, Middle East African Journal of Ophthalmology. 2009;16(4112-115), Other attributes of failed or failing blebs may include cystic appearance and/or changes in vascularization and/or scar tissue and/or thinning of the conjunctiva overlaying the bleb and/or a tense bleb and/or other observable or measurable changes as may be included in either the Indiana Bleb Appearance Grading Scale or Moorfields Bleb Grading System. Other functional determinates of failed or failing blebs or glaucoma surgery may include increased IOP, or IOP that has not decreased sufficiently.

Minimally Invasive Glaucoma Surgery (MIGS): MIGS is a recent innovation in the surgical treatment of glaucoma developed to minimize the complications from tubes and trabeculectomy. MIGS is a term applied to the widening range of implants, devices, and techniques that seek to lower intraocular pressure with less surgical risk than the more established procedures. In most cases, conjunctiva-involving devices require a subconjunctival bleb to receive the fluid and allow for its extraocular resorption. Flow-controlled conjunctiva-involving devices typically attempt to control flow and lower IOP to normal pressure and also minimizing hypotony (too low pressure in the eye) by applying Poiseuille's law of laminar flow to create a tube that is sufficiently long and narrow to restrict and control outflow. Some MIGS devices include Flow Controlled Stents, microshunts to Shlemm's Canal, Suprachoroidal Devices, and devices for Trabeculotomy. Examples of microshunts to Schlemm's Canal include iStent® (Glaukos®) and Hydrus™ (Ivantis). Examples of suprachoroidal devices include CyPass® (Alcon), Solx® gold shunt (Solx), and iStent Supra® (Glaukos). An example of a trabeculotomy device includes the Trabectome® (NeoMedix) electrocautery device.

Planning Treatment Volume or Planning Target Volume (PTV): An area or volume that encloses all the tissue intended for irradiation. The PTV includes the clinical target volume or clinical treatment volume (CTV).

Radioactive isotope, radionuclide. radioisotope: An element that has an unstable nucleus and emits radiation during its decay to a stable form. There may be several steps in the decay from a radioactive to a stable nucleus. There are four types of radioactive decay: alpha, beta negative, beta positive, and electron capture. Gamma rays can be emitted by the daughter nucleus in a de-excitation following the decay process. These emissions are considered ionizing radiation because they are powerful enough to liberate an electron from another atom.

Therapeutic radionuclides can occur naturally or can be artificially produced, for example by nuclear reactors or particle accelerators. Radionuclide generators are used to separate daughter isotopes from parent isotopes following natural decay.

Non-limiting examples of radioactive isotopes following one of the four decay processes are given herein: (1) Alpha decay: radium 226, americium 241; (2) Beta minus: iridium 192, cesium 137, phosphorous 32 (P-32), strontium 90 (Sr-90), yttrium 90 (Y-90), ruthenium 106, rhodium-106; (3) Beta positive: fluorine 18; (4) Electron capture: iodine 125, palladium 106. Examples of gamma emission include iridium 192 and cesium 137.

Half-life is defined as the time it takes for one-half of the atoms of a radioactive material to disintegrate. Half-lives for various radioisotopes can range from a few microseconds to billions of years.

The term activity in the radioactive-decay processes refers to the number of disintegrations per second. The units of measure for activity in a given source are the curie (Ci) and becquerel (Bq). One (1) Becquerel (Bq) is one disintegration per second.

An older unit is the Curie (Ci), wherein one (1) Ci is 3.7×10¹⁰ Bq.

The term “beta radiation source” or “source of beta radiation” can refer to the term “radioisotope.” In any of the methods or compositions here, the radioisotope or source of beta radiation may comprise Strontium-90 (Sr-90), Phosphorus-32 (P-32), Ruthenium 106 (Ru-106), Yttrium 90 (Y-90), or a combination thereof.

Radionuclide Brachytherapy Source (RBS) (see also Brachytherapy): According to the US Federal Code of Regulations, a Radionuclide Brachytherapy Source (RBS) is “a device that consists of a radionuclide what may be enclosed in a sealed container made of gold, titanium, stainless steel, or platinum and intended for medical purposes to be placed onto a body surface or into a body cavity or tissue as a source of nuclear radiation for therapy.” Other forms of brachytherapy sources are also used in practice. For example, a commercially available conformal source is a flexible, thin film made of a polymer chemically bound to Phosphorous-32 (P-32). Another product is the TheraSphere, a radiotherapy treatment for hepatocellular carcinoma (HCC) that consists of millions of microscopic, radioactive glass microspheres (20-30 micrometers in diameter) containing Yttrium-90. Other forms of brachytherapy employ x-ray generators as sources instead of radioisotopes.

Sclerostomy: A procedure in which the surgeon makes a small opening in the sclera to reduce intraocular pressure (IOP), usually in patients with open-angle glaucoma. It is classified as a type of glaucoma filtering surgery. Minimally invasive micro sclerostomy (MIMS, Sanoculis) is a recent innovative technique that combines the mechanism of conventional trabeculectomy and simple needling. In the course of the surgery, a sclero-corneal drainage channel is created. The MIMS procedure can be performed ab externo by creating a sclero-corneal channel to drain the aqueous humor from the anterior chamber to the subconjunctival space. The channel created with MIMS is designed to obtain a controlled fluid flow. Laser sclerostomy can be performed in a less invasive manner than standard filtering surgery. Other studies have explored the use of laser energy of varying wavelengths, properties, and tissue interaction to create thermal sclerostomies. Several methods deliver laser energy by mirrored contact lenses to the internal face of the filtration angle or by fiberoptic cables for ab interno or ab externo sclerostomy formation.

Trabeculectomy: A procedure wherein a small hole is made in the sclera and is covered by a thin trap-door. Aqueous humor drains through the trap door to a bleb, As an example, in some trabeculectomy procedures, an initial pocket is created under the conjunctiva and Tenon's capsule and the wound bed is treated with mitomycin C soaked sponges using a “fornix-based” conjunctival incision at the corneoscleral junction, A partial thickness scleral flap with its base at the corneoscleral junction after cauterization of the flap area is created. Further, a window opening is created under the flap with a Kelly-punch or a Khaw Descemet Membrane Punch to remove a portion of the sclera, Schlemm's canal, and the trabecular meshwork to enter the anterior chamber. An iridectomy is done in many cases to prevent future blockage of the sclerostomy. The scleral flap is then sutured loosely back in place with several sutures, The conjunctiva is closed in a watertight fashion at the end of the procedure.

Trans-scleral Drainage Devices: Devices that shunt aqueous humor from the anterior chamber to a subconjunctival reservoir. As an example, the EX-PRESS® Glaucoma Filtration Device channels aqueous humor through a secure lumen to a half-thickness scleral flap, creating a subconjunctival filtration bleb. The device's lumen provides a standardized opening for aqueous humor flow while also providing some resistance, which appears to add further stability to the anterior chamber during surgery and the early post-op period.

Treat, Treatment, Treating: These terms refer to both therapeutic treatments, e.g., elimination of a disease, disorder, or condition, and prophylactic or preventative measures, e.g., preventing or slowing the development of a disease or condition, reducing at least one adverse effect or symptom of a disease, condition, or disorder, etc. Treatment may be “effective” if one or more symptoms or clinical markers are reduced as that term is defined herein. Alternatively, a treatment may be “effective” if the progression of a disease is reduced or halted, That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable, “Treatment” can also mean prolonging survival as compared to expected survival if not receiving treatment. Those in need of treatment include those already diagnosed with a particular disease, disorder, or condition, as well as those likely to develop a particular disease, disorder, or condition due to genetic susceptibility or other factors.

Valves: Devices that can be used for glaucoma treatment, wherein instead of using a natural bleb, these devices use a synthetic reservoir (or plate), which is implanted under the conjunctiva to allow flow of aqueous fluid. Valve devices include the Baerveldt® implant (Pharmacia Co.), the Ahmed® glaucoma valve (New World Medical), the Krupin-Denver eye valve to disc implant (E. Benson Hood Laboratories), and the Molteno® and Molteno3® drainage devices (Molteno® Ophthalmic Ltd.).

### DETAILED DESCRIPTION OF THE INVENTION

The present invention provides methods and system for achieving a healthy intraocular pressure following combined glaucoma and cataract surgery. For example, the methods and systems herein may help effectively maintain functioning drainage blebs and/or drainage channels (e.g., help avoid scar formation or wound reversion; inhibit or reduce the fibrogenesis and/or inflammation in the blebs or holes, etc.) are associated with glaucoma procedures.

The methods feature applying a therapeutic dose of beta radiation to the target site (e.g., drainage site or other appropriate site) at or around the time of combined cataract surgery and glaucoma surgery (e.g., implantation of a drainage device, e.g., MIGS implantation), e.g., before glaucoma surgery, after glaucoma surgery, before cataract surgery, after cataract surgery, etc. The methods herein may also feature applying a drug to the eye, e.g., to the target, to an area near the target, etc. Non-limiting examples of drugs include mitomycin C, 5 fluorouracil, an anti-VEGF composition, and other appropriate compositions.

The methods allow for achieving a healthy intraocular pressure (IOP). In some embodiments, the methods herein allow for achieving an IOP of 10 mmHg or less. In some embodiments, the methods herein allow for achieving an IOP of 10 mmHg. In some embodiments, the methods herein allow for achieving an IOP of 11 mmHg. In some embodiments, the methods herein allow for achieving an IOP of 12 mmHg. In some embodiments, the methods herein allow for achieving an IOP of 13 mmHg. In some embodiments, the methods herein allow for achieving an IOP of 14 mmHg. In some embodiments, the methods herein allow for achieving an IOP of 15 mmHg. In some embodiments, the methods herein allow for achieving an IOP from 10-12 mmHg. In some embodiments, the methods herein allow for achieving an IOP from 10-13 mmHg. In some embodiments, the methods herein allow for achieving an IOP from 10-14 mmHg. In some embodiments, the methods herein allow for achieving an IOP from 10-15 mmHg. In some embodiments, the methods herein allow for achieving an IOP from 9-12 mmHg, In some embodiments, the methods herein allow for achieving an IOP from 9-15 mmHg.

As used herein, the term “drainage device” refers to any or a combination of the general and specific approaches for draining aqueous humor, such as the therapeutics and devices described herein, including but not limited to minimally invasive glaucoma surgery (MIGS) devices and surgery, that are employed to reduce Intraocular Pressure by means of a surgical intervention with a device.

Various glaucoma drainage procedures and devices, including trabeculectomy, drainage tubes, and devices used for Minimally Invasive Glaucoma Surgery (MIGS), are described herein. For the purposes of the invention, other surgical innovations and/or devices in addition to those described above may be included in the scope of the invention and described and labeled as MIGS. For example, techniques and devices that may alternatively be described as Moderately Invasive Glaucoma Surgery or Augmented Incisional Surgery is also included in the present invention.

### Isotopes and Radioactivity

The US Nuclear Regulatory Commission (USNRC) (https://www.nrc.gov/about-nrc/radiation/health-effects/measuring-radiation.html) defines radioactivity as “the amount of ionizing radiation released by a material. Whether it emits alpha or beta particles, gamma rays, x-rays, or neutrons, a quantity of radioactive material is expressed in terms of its radioactivity (or simply its activity), which represents how many atoms in the material decay in a given time period. The units of measure for radioactivity are the curie (Ci) and becquerel (Bq).” Activity in a radioactive-decay process is defined as the number of disintegrations per second, or the number of unstable atomic nuclei that decay per second in a given sample. Activity is expressed in the International System of Units by the becquerel (abbreviated Bq), which is exactly equal to one disintegration per second. Another unit that may be used is the Curie, wherein one curie is approximately the activity of 1 gram of radium and equals (exactly) 3.7×10¹⁰ becquerel. The specific activity of radionuclides is relevant when it comes to select them for production for therapeutic pharmaceuticals.

By the USNRC definition, absorbed dose is defined as the amount of radiation absorbed, e.g., the amount of energy that radioactive sources deposit in materials through which they pass or the concentration of energy deposited in tissue as a result of an exposure to ionizing radiation. The absorbed dose is equal to the radiation exposure (ions or Ci/kg) of the radiation beam multiplied by the ionization energy of the medium to be ionized. Typically, the units for absorbed dose are the radiation absorbed dose (rad) and gray (Gy). Gy is a unit of ionizing radiation dose defined as the absorption of one joule of radiation energy per kilogram of matter. The rad has generally been replaced by the Gy in SI derived units. 1 Gy is equivalent to 100 rad.

Radionuclide generators are devices that produce a useful short-lived medical radionuclide (known as “daughter” products) from the radioactive transformation of a long-lived radionuclide (called a “parent”). By having a supply of parent on hand at a facility, the daughter is continually generated on site. The generator permits ready separation of the daughter radionuclide from the parent. One of the most widely used generator devices (often referred as a “cow”) is the technetium 99 generator. It allows the extraction of the metastable isotope 99 mTc of technetium from a source of decaying molybdenum-99. 99 Mo has a half-life of 66 hours and can be easily transported over long distances to hospitals where its decay product technetium-99m (with a half-life of only 6 hours, inconvenient for transport) is extracted and used for a variety of nuclear medicine procedures, where its short half-life is very useful.

Generators can also be constructed for supply of other daughter radioisotopes. Ruthenium 106 (Ru-106) is a commercially available radioisotope with a half-life of 668-373 days, making it a good candidate for a parent isotope in a cow or generator. The decay of Ru-106 to rhodium-106 (Rh-106) produces only a low energy beta of 39 Key that is not useful for therapy. However, Rh-106 has an energetic beta decay useful for brachytherapy: Rh-106 has a half-life of 30 seconds and decays by beta emission to palladium 106 (Pd-106) with a maximum decay energy of 3.541 Mev and an average energy of 96.9 Key. As an example, in some embodiments, the present invention features a device loaded from a Ruthenium-106 cow with an activity of rhodium-106 providing for the full prescribed dose. The device can be applied to the target volume to deliver the full activity of its contents. For example, the device may be placed over the target lesion for 10 half-lives (300 seconds), delivering all its radioactive energy and consuming the rhodium-106, depleting it to palladium.

In some embodiments, the present invention features the use of Ru-106 in secular equilibrium with Rh-106. Ru-106 decays by beta radiation to Rh-106. The two isotopes are in secular equilibrium with the decay rate of the combined source controlled by the Ru-106 parent but with the therapeutic beta radiations emanating from the daughter Rh-106.

Yttrium-90 is commercially available from Strontium-90 cows. As another example, in some embodiments, the present invention features the use of Yttrium-90 with a half-life of 64 hours. Y-90 decays to Zirconium 90 (Zr-90), a stable isotope, along three different routes via beta emission, wherein 99.985% of the time it decays with a maximum beta particle energy of 2.2801 MeV and a mean beta particle energy of 0.9337 MeV, or approximately or 1.5×10-13 joules. The other minor decay paths produce additional low energy gamma-rays, and electrons. Compared to the dominant path, the radiation doses from these paths are clinically negligible.

Currently, strontium-90 is also commercially available. As another example, in some embodiments, the present invention features the use of Strontium 90 (Sr-90) in secular equilibrium with Yttrium 90 (Y-90). Strontium 90 (Sr-90) decays by beta radiation to Yttrium 90 (Y-90). The parent Sr-90 isotope has a half-life of 28.79 years. The daughter Y-90 isotope has a half-life of 64.0 hours. The two isotopes are in secular equilibrium with the decay rate of the combined source controlled by the Sr-90 parent but with the therapeutic beta radiations emanating from the daughter Y-90 with maximum energy of 2.28 MeV and an average energy of 934 keV.

The Planning Target Volume (PTV) or Planning Treatment Volume (PTV) is a geometrical concept introduced for radiation treatment planning. The PTV is used to ensure that the prescribed dose is actually delivered to all parts of the target tissue. Without limiting the invention to any particular surgical practice, a medical journal article details the surgical creation of the bleb in which “the surgeon dissects backward with Westcott scissors to make a pocket approximately 10 to 15 mm posteriorly and sufficiently wide to accommodate the antimetabolite sponges”. In this example, the surgeon opened the potential space under the conjunctiva and Tenon's capsule creating an approximately 10 to 15 mm diameter bleb site. As an example, it would follow that the Target Volume could be defined as a disk of diameter 15 mm and depth of 0.3 mm, containing the conjunctiva and Tenon's capsule tissue.

For example, a prescription dose of brachytherapy of 10 Gray (1000 cGy) is 10 joules/kg absorbed dose throughout the Target Volume. Measurements have suggested a model Sr-90/Y-90 RBS with Activity of 1.48 GBq produces a surface dose rate of approximately 0.20 Gy per second. To deliver a dose of 10 Gy to the Target Volume would require an irradiation time of 50 seconds. The number nuclei that decay during this 50 second treatment would be 1.48×10⁹ Bq (disintegrations per second)×50 seconds=7.4×10¹⁰.

### Targets of the Eye

As previously discussed, the present invention provides methods and systems for applying beta radiation to a treatment area or target of the eye. In some embodiments, the target is a site of the bleb in an eye being treated for glaucoma with a MIGS implant or MIGS procedure. In some embodiments, the target is a site of the bleb in an eye treated with a trabeculectomy. In some embodiments, the target is a site of the bleb in an eye treated with minimally invasive micro sclerostomy (MIMS), In some embodiments, the target is a site of the hole in an eye treated with MIMS. In some embodiments, the target is a site of the implant that is surgically inserted into the eye for the purpose of treating glaucoma. In some embodiments, the target is a site of the eye associated with pterygium.

In some embodiments, the target comprises an entire bleb. In some embodiments, the target comprises a portion of a bleb. In some embodiments, the target area surrounds an end of the MIGS implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel.

In some embodiments, the target area is the entire bleb, e.g., the perimeter of the bleb, the center of the bleb, and the portions of the bleb in between the perimeter and the center. In some embodiments, the target area is the perimeter of the bleb, e.g., a ring-shaped target area. In some embodiments, the target is the perimeter of the bleb and a portion of the bleb next to the perimeter, e.g., the target may be annulus-shaped. hi some embodiments, the target is a portion of the bleb in between the center and the perimeter. In some embodiments, the target is at least a portion of the center of the bleb. The present invention is not limited to the aforementioned descriptions of target areas. For example, in certain embodiments, the target is (or includes) tissue surrounding the rim of a drainage channel.

In some embodiments, the target is a target other than that associated with MIGS/MIMS/trabeculectomy. In some embodiments, the ophthalmic target is other targets than those associated with glaucoma drainage surgery. In some embodiments the target is inflammation, autoimmune mediated pathologies, or vascular pathologies of the eye, In some embodiments, the target comprises macrophages, In some embodiments, the target comprises fibroblasts. In some embodiments, the target comprises endothelial cells, In some embodiments, the target is associated with infections (for example, Herpes Simplex Keratitis or Tuberculous sclerokeratitis), Corneal ulcerations (for example, Moorens), Allergic disorders (for example, Vernal), benign or malignant Tumors (for example, Squamous Cell Carcinoma) or benign growths (for example, papillomas), Degenerations (for example, pterygium), Cicitarising disease (for example, pemphigoid), Inflammations (for example, meibomian gland), ocular manifestations of Stevens-Johnson syndrome, Drug-induced cicatrizing conjunctivitis, Ligneous conjunctivitis, Corneal Vascularization, Pterygia, Vernal Catarrh, Small papillomas of the eyelid, limbal carcinoma, ocular malignant melanoma, nevus pigmentosus of the conjunctiva, hemangioma, chalazion. In some embodiments, the target is in the orbit of the eye. The present invention includes other ophthalmic indications and is not limited to the aforementioned targets.

The system of the present invention delivers a dose of radiation to a target area or treatment area. The target area or treatment area may be a plane of a particular size (e.g., diameter) at a particular depth (e.g. a distance from the outer surface of the applicator, a distance from the surface of the eye, a distance from the top of the bleb, a distance from the RBS, etc.) within the tissue being exposed to beta radiation.

In certain embodiments, the target plane has a diameter of about 2 mm. In certain embodiments, the target plane has a diameter of about 3 mm. In certain embodiments, the target plane has a diameter of about 4 mm. In certain embodiments, the target plane has a diameter of about 5 mm. In certain embodiments, the target plane has a diameter of about 6 mm, In certain embodiments, the target plane has a diameter of about 7 mm. In certain embodiments, the target plane has a diameter of about 8 mm. In certain embodiments, the target plane has a diameter of about 9 mm. In certain embodiments, the target plane has a diameter of about 10 mm. In certain embodiments, the target plane has a diameter of about 11 mm. In certain embodiments, the target plane has a diameter of about 12 mm. In certain embodiments, the target plane has a diameter from 10 to 14 mm. In certain embodiments, the target plane has a diameter from 6 to 10 mm. In certain embodiments, the target plane has a diameter from 5 to 12 mm. In certain embodiments, the target plane has a diameter from 6 to 12 mm, In certain embodiments, the target plane has a diameter from 8 to 10 mm. In certain embodiments, the target plane has a diameter from 8 to 12 mm. In certain embodiments, the target plane has a diameter from 6 to 8 mm. In certain embodiments, the target plane has a diameter from 7 to 10 mm. In certain embodiments, the target plane has a diameter from 8 to 11 mm. In certain embodiments, the target plane has a diameter from 9 to 11 mm. In certain embodiments, the target plane has a diameter from 9 to 12 mm, The present invention is not limited to the aforementioned dimensions of the target surface.

In certain embodiments, the target plane is a distance from 0 to 700 microns, e.g., from the outer surface of the applicator (e.g., portion of the applicator that contacts the eye tissue), from the surface of the eye, from the top of the bleb, from the RBS, etc. In certain embodiments, the target plane is a distance from 0 to 100 microns, e.g., from the outer surface of the applicator (e.g., portion of the applicator that contacts the eye tissue), from the surface of the eye, from the top of the bleb, from the RBS, etc. In certain embodiments, the target plane is a distance from 100 to 200 microns, e,g., from the outer surface of the applicator (e.g., portion of the applicator that contacts the eye tissue), from the surface of the eye, from the top of the bleb, from the RBS, etc. In certain embodiments, the target plane is a distance from 200 to 400 microns, e.g., from the outer surface of the applicator (e.g., portion of the applicator that contacts the eye tissue), from the surface of the eye, from the top of the bleb, from the RBS, etc. In certain embodiments, the target plane is a distance from 200 to 600 microns, e.g., from the outer surface of the applicator (e.g., portion of the applicator that contacts the eye tissue), from the surface of the eye, from the top of the bleb, from the RBS, etc. In certain embodiments, the target plane is a distance from 400 to 600 microns, e.g., from the outer surface of the applicator (e.g., portion of the applicator that contacts the eye tissue), from the surface of the eye, from the top of the bleb, from the RBS, etc.

In certain embodiments, the dose across the particular target plane on or within the target varies by no more than 10% of the maximum dose. In certain embodiments, the dose across the particular plane on or within the target varies by no more than 15% of the maximum dose. In certain embodiments, the dose across the particular plane on or within the target varies by no more than 20% of the maximum dose. In certain embodiments, the dose across the particular plane on or within the target varies by no more than 30% of the maximum dose. In certain embodiments, the dose at any point on the target plane of the treatment area is within 10% of a dose at any other point on the target plane of the treatment area. In certain embodiments, the dose at any point on the target plane of the treatment area is within 20% of a dose at any other point on the target plane of the treatment area. In certain embodiments, the dose at any point on the target plane of the treatment area is within 30% of a dose at any other point on the target plane of the treatment area. In certain embodiments, the dose at any point on the target plane of the treatment area is within 40% of a dose at any other point on the target plane of the treatment area. In certain embodiments, the dose at any point on the target plane of the treatment area is within 50% of a dose at any other point on the target plane of the treatment area.

In some embodiments, a dose of radiation is delivered to a plurality of points on the target plane, wherein a dose received by one point on the target plane is within 50% of the dose received by any other point on the target plane. In some embodiments, a dose of radiation is delivered to a plurality of points on the target plane, wherein a dose received by one point on the target plane is within 40% of the dose received by any other point on the target plane. In some embodiments, a dose of radiation is delivered to a plurality of points on the target plane, wherein a dose received by one point on the target plane is within 30% of the dose received by any other point on the target plane. In some embodiments, a dose of radiation is delivered to a plurality of points on the target plane, wherein a dose received by one point on the target plane is within 20% of the dose received by any other point on the target plane. In some embodiments, a dose of radiation is delivered to a plurality of points on the target plane, wherein a dose received by one point on the target plane is within 15% of the dose received by any other point on the target plane. In some embodiments, a dose of radiation is delivered to a plurality of points on the target plane, wherein a dose received by one point on the target plane is within 10% of the dose received by any other point on the target plane.

### Application of Beta Radiation

The methods and systems of the present invention deliver a particular radiation dose to the target, e.g., to a plane within the target (e.g., a plane of a certain size at a certain depth representing a portion of the treatment area (e.g., PTV)).

In some embodiments, the methods and systems deliver a radiation dose of 1000 cGy (10 Gy) to the target. In some embodiments, the methods and systems deliver a radiation dose of 900 cGy to the target. In some embodiments, the methods and systems deliver a radiation dose of 800 cGy to the target. In some embodiments, the methods and systems deliver a radiation dose of 750 cGy to the target. In some embodiments, the methods and systems deliver a radiation dose of 600 cGy to the target. In some embodiments, the methods and systems deliver a radiation dose of 500 cGy to the target. In some embodiments, the methods and systems deliver a radiation dose of 400 cGy to the target. In some embodiments, the methods and systems deliver a radiation dose of 300 cGy to the target. In some embodiments, the methods and systems deliver a radiation dose of 200 cGy to the target. In some embodiments, the methods and systems deliver a radiation dose of 100 cGy to the target. In some embodiments, the methods and systems deliver a radiation dose of 50 cGy to the target. In some embodiments, the methods and systems deliver a radiation dose of 1100 cGy to the target. In some embodiments, the methods and systems deliver a radiation dose of 1200 cGy to the target. In some embodiments, the methods and systems deliver a radiation dose of 1300 cGy to the target. In some embodiments, the methods and systems deliver a radiation dose of 1500 cGy to the target. In some embodiments, the methods and systems deliver a radiation dose from 600 cGy and 1500 cGy to the target. In some embodiments, the methods and systems deliver a radiation dose from 50 cGy to 100 cGy. hi some embodiments, the methods and systems deliver a radiation dose from 100 cGy to 150 cGy. In some embodiments, the methods and systems deliver a radiation dose from 150 cGy to 200 cGy. In some embodiments, the methods and systems deliver a radiation dose from 200 cGy to 250 cGy. In some embodiments, the methods and systems deliver a radiation dose from 250 cGy to 300 cGy. In some embodiments, the methods and systems deliver a radiation dose from 300 cGy to 350 cGy. In some embodiments, the methods and systems deliver a radiation dose from 350 cGy to 400 cGy, In some embodiments, the methods and systems deliver a radiation dose from 400 cGy to 450 cGy. In some embodiments, the methods and systems deliver a radiation dose from 450 cGy to 500 cGy. In some embodiments, the methods and systems deliver a radiation dose from 500 cGy to 550 cGy. In some embodiments, the methods and systems deliver a radiation dose from 550 cGy to 600 cGy. In some embodiments, the methods and systems deliver a radiation dose from 600 cGy to 650 cGy. In some embodiments, the methods and systems deliver a radiation dose from 650 cGy to 700 cGy, In some embodiments, the methods and systems deliver a radiation dose from 700 cGy to 750 cGy. In some embodiments, the methods and systems deliver a radiation dose from 750 cGy to 800 cGy. In some embodiments, the methods and systems deliver a radiation dose from 800 cGy to 850 cGy. In some embodiments, the methods and systems deliver a radiation dose from 850 cGy to 900 cGy. In some embodiments, the methods and systems deliver a radiation dose from 900 cGy to 950 cGy. In some embodiments, the methods and systems deliver a radiation dose from 950 cGy to 1000 cGy. In some embodiments, the methods and systems deliver a radiation dose from 1000 cGy to 1050 cGy. In some embodiments, the methods and systems deliver a radiation dose from 1050 cGy to 1100 cGy. In some embodiments, the methods and systems deliver a radiation dose from 1100 cGy to 1150 cGy. In some embodiments, the methods and systems deliver a radiation dose from 1150 cGy to 1200 cGy. In some embodiments, the methods and systems deliver a radiation dose from 1200 cGy to 1250 cGy. In some embodiments, the methods and systems deliver a radiation dose from 1250 cGy to 1300 cGy. In some embodiments, the methods and systems deliver a radiation dose from 1300 cGy to 1350 cGy. In some embodiments, the methods and systems deliver a radiation dose from 1350 cGy to 1400 cGy. In some embodiments, the methods and systems deliver a radiation dose from 1400 cGy to 1450 cGy. In some embodiments, the methods and systems deliver a radiation dose from 1450 cGy to 1500 cGy. In some embodiments, the methods and systems deliver a radiation dose from 1500 cGy to 1550 cGy. In some embodiments, the methods and systems deliver a radiation dose from 1550 cGy to 1600 cGy. In some embodiments, the methods and systems deliver a radiation dose from 1600 cGy to 1800 cGy. In some embodiments, the methods and systems deliver a radiation dose from 1800 cGy to 2000 cGy. In some embodiments, the methods and systems deliver a radiation dose of 600, 650, 700, 750, 800, 850, 900, 950, 1000, 1050, 1100, 1150, 1200,1250, 1300, 1350, 1400, 1450, or 1500 cGy to the target. In some embodiments, the methods and systems deliver a radiation dose of 1500 to 3200 cGy. In some embodiments, the methods and systems deliver a radiation dose of 3200 to 8000 cGy. In some embodiments, the methods and systems deliver a radiation dose of 8000 cGy to 10000 cGy. In some embodiments, the methods and systems deliver a radiation dose of greater than 10000 cGy.

The doses cited herein may refer to the doses at a particular depth from the surface of the device, for example at a depth of 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, etc.

In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 10% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 15% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 20% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 25% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 30% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 35% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 40% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 45% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 50% of a dose at any other point on the target.

In some embodiments, the methods and systems deliver the prescribed dose in a time from 10 seconds to 20 minutes. In some embodiments, the methods and systems deliver the prescribed dose in a time from 20 seconds and 10 minutes. In some embodiments, the methods and systems deliver the prescribed dose in a time from 20 seconds to 60 seconds. In some embodiments, the methods and systems deliver the prescribed dose in a time from 30 seconds to 90 seconds. In some embodiments, the methods and systems deliver the prescribed dose in a time from 60 seconds to 90 seconds, In some embodiments, the methods and systems deliver the prescribed dose in a time from 90 seconds to 2 minutes, In some embodiments, the methods and systems deliver the prescribed dose in a time from 2 minutes to 3 minutes.

In some embodiments, the methods and systems deliver the prescribed dose in a time from 3 minutes to 4 minutes. In some embodiments, the methods and systems deliver the prescribed dose in a time from 3 minutes to 5 minutes. In some embodiments, the methods and systems deliver the prescribed dose in a time from 3 minutes to 6 minutes. In some embodiments, the methods and systems deliver the prescribed dose in a time from 4 minutes to 5 minutes. hi some embodiments, the methods and systems deliver the prescribed dose in a time from 4 minutes to 6 minutes. In some embodiments, the methods and systems deliver the prescribed dose in a time from 5 minutes to 6 minutes. In some embodiments, the methods and systems deliver the prescribed dose in a time from 6 minutes to 7 minutes. In some embodiments, the methods and systems deliver the prescribed dose in a time from 7 minutes to 8 minutes. In some embodiments, the methods and systems deliver the prescribed dose in a time from 8 minutes to 9 minutes. In some embodiments, the methods and systems deliver the prescribed dose in a time from 9 minutes to 10 minutes, In some embodiments, the methods and systems deliver the prescribed dose in a time from 10 minutes to 12 minutes. In some embodiments, the methods and systems deliver the prescribed dose in a time from 12 minutes to 15 minutes. In some embodiments, the methods and systems deliver the prescribed dose in a time from 15 minutes to 20 minutes.

In some embodiments, the methods and systems deliver the prescribed dose within 5 seconds. In some embodiments, the methods and systems deliver the prescribed dose within 10 seconds. In some embodiments, the methods and systems deliver the prescribed dose within 15 seconds, In some embodiments, the methods and systems deliver the prescribed dose within 20 seconds. In some embodiments, the methods and systems deliver the prescribed dose within 25 seconds. In some embodiments, the methods and systems deliver the prescribed dose within 45 seconds. In some embodiments, the methods and systems deliver the prescribed dose within 60 seconds. In some embodiments, the methods and systems deliver the prescribed dose within 90 seconds. In some embodiments, the methods and systems deliver the prescribed dose within 2 minutes, In some embodiments, the methods and systems deliver the prescribed dose within 3 minutes. In some embodiments, the methods and systems deliver the prescribed dose within 4 minutes. In some embodiments, the methods and systems deliver the prescribed dose within 5 minutes. In some embodiments, the methods and systems deliver the prescribed dose within 6 minutes. In some embodiments, the methods and systems deliver the prescribed dose within 7 minutes. In some embodiments, the methods and systems deliver the prescribed dose within 8 minutes. In some embodiments, the methods and systems deliver the prescribed dose within 9 minutes, In some embodiments, the methods and systems deliver the prescribed dose within 10 minutes. In some embodiments, the methods and systems deliver the prescribed dose within 11 minutes. In some embodiments, the methods and systems deliver the prescribed dose within 12 minutes. In some embodiments, the methods and systems deliver the prescribed dose within 13 minutes. In some embodiments, the methods and systems deliver the prescribed dose within 14 minutes. In some embodiments, the methods and systems deliver the prescribed dose within 15 minutes. In some embodiments, the methods and systems deliver the prescribed dose within 16 minutes, In some embodiments, the methods and systems deliver the prescribed dose within 17 minutes. In some embodiments, the methods and systems deliver the prescribed dose within 18 minutes. In some embodiments, the methods and systems deliver the prescribed dose within 19 minutes. In some embodiments, the methods and systems deliver the prescribed dose within 20 minutes. In some embodiments, the methods and systems deliver the prescribed dose in a time frame greater than 20 minutes.

In some embodiments, a dose (e.g., a prescribed dose) may be delivered in a single application. In other embodiments, a dose (e.g., a prescribed dose) may be fractionated and applied in multiple applications. For example, in some embodiments, radiation (e.g., a prescribed dose) may be applied over the course of 2 applications. In some embodiments, radiation (e.g., a prescribed dose) may be applied over the course of 3 applications. In some embodiments, radiation (e.g., a prescribed dose) may be applied over the course of 4 applications. In some embodiments, radiation (e.g., a prescribed dose) may be applied over the course of 5 applications. In some embodiments, radiation (e.g., a prescribed dose) may be applied over the course of more than 5 applications. In some embodiments, radiation (e.g., a prescribed dose) may be applied over the course of 20 applications. In some embodiments, radiation (e.g., a prescribed dose) may be applied over the course of more than 20 applications.

Each application may deliver an equal sub-dose. In some embodiments, one or more of the sub-doses are different. For example, one or more of the sub-doses may be different so as to increase or decrease with each additional application.

According to one embodiment, a dose of radiation may be applied prior to the treatment procedure, e.g., surgery for implantation of a device, e.g., MIGS device, or other appropriate glaucoma procedure, e.g., MIMS. For example, in some embodiments, a dose of radiation may be applied one or more days prior to a surgery (e.g., insertion of a device, MIMS, etc.). In some embodiments, a dose of radiation may be applied within a 24-hour prior before a surgery (e.g., insertion of a device). In some embodiments, a dose of radiation may be applied just prior to a surgery (e.g., insertion of a device, MIMS, etc.), e.g., 1 hour before, 30 minutes before, 15 minutes before, 5 minutes before 1 minute before, etc. In some embodiments, a dose of radiation may be applied during a procedure, e.g., for implantation of a device. In some embodiments, a dose of radiation may be applied right after a surgery (e.g., implantation of a device (e.g., MIGS device), MIMS, etc.), e.g., within 1 minute, 2 minutes, 3 minutes, 5 minutes, 10 minutes, etc.). In some embodiments, a dose of radiation may be applied before an incision is made into the conjunctiva. In some embodiments, a dose of radiation may be applied after an incision is made into the conjunctiva. In other embodiments, a dose of radiation may be applied after a surgery (e.g., insertion of a device). In some embodiments, a dose of radiation may be applied within a 24-hour period after a surgery (e.g., insertion of a device). In some embodiments, a dose of radiation may be applied within one to two days after a surgery (e.g., insertion of a device). In some embodiments, a dose of radiation may be applied within 2 or more days after a surgery (e.g., insertion of a device). In some embodiments the dose may be applied any time after the glaucoma surgery. In some embodiments, the dose is applied months or years after the glaucoma surgery. For example, a dose may be given to patients that did not receive a dose during surgery but at a future date have scar or needling procedures to break up scar tissue.

### Methods

The present invention features methods and systems for applying a therapeutic amount of beta radiation to a treatment area, such as a target area of a bleb for draining aqueous humor, such as but not limited to a bleb associated with a Minimally Invasive Glaucoma Surgery (MIGS) implant or foreign body inserted between an anterior chamber of the eye and a subconjunctival space of the eye or between the anterior chamber of the eye and a space between the conjunctiva and Tenon's capsule, in combination with combined glaucoma and cataract surgery. The methods and systems herein may be used to apply beta radiation to a target area in the eye to help maintain functioning blebs and/or drainage holes arising from glaucoma drainage procedures or surgeries, to help avoid scar formation or wound reversion, to inhibit or reduce fibrogenesis and/or inflammation in the blebs or surrounding areas, to treat glaucoma, to reduce intraocular pressure (IOP), to achieve and/or maintain a healthy IOP, for causing cell cycle arrest in fibroblasts on the Tenon's capsule, to enhance function of a drainage device such as a MIGS implant, etc. The present invention is not limited to the applications disclosed herein.

The methods may feature the application of the therapeutic amount of beta radiation from a radioisotope to a target area of the eye using an applicator system.

In some embodiments, the methods comprise performing glaucoma surgery, which forms a bleb for draining aqueous humor. For example, the method may comprise implanting a Minimally Invasive Glaucoma Surgery (MIGS) implant within the eye, wherein the implant causes formation of a bleb (e.g., in the subconjunctival space of the eye, in a space between the conjunctiva and Tenon's capsule); the bleb functions to drain aqueous humor. In certain embodiments, the implant is inserted trans-sclerally, between an anterior chamber of the eye and a subconjunctival space of the eye, between the anterior chamber of the eye and a space between the conjunctiva and Tenon's capsule, etc.

In some embodiments, the methods comprise performing cataract surgery.

The methods feature applying a therapeutic dose of beta radiation to the target site (e.g., drainage site or other appropriate site) at or around the time of combined cataract surgery and glaucoma surgery (e.g., implantation of a drainage device, e.g., MIGS implantation), e.g., before glaucoma surgery, after glaucoma surgery, before cataract surgery, after cataract surgery, etc. For example, the method may comprise applying the beta radiation prior to insertion of a MIGS implant, prior to incision of the conjunctive, prior to creation of a hole associated with MIMS, etc, In some embodiments, the method comprises applying the beta radiation after insertion of a MICS implant, prior to incision of the conjunctive, prior to creation of a hole associated with MIMS, etc.

The methods herein may also feature applying a drug to the eye, e.g., to the target, to an area near the target, to a site of a drainage device or implant, to the side of the bleb, to a different part of the eye, etc. Non-limiting examples of drugs include mitomycin C, 5 fluorouracil, an anti-VEGF composition, and other appropriate compositions. In some embodiments, the drug is administered before, during, and/or after a surgical procedure.

As previously discussed, the beta radiation may be applied via a radionuclide brachytherapy source (RBS). The RBS may be applied to the target via an applicator. As previously discussed, in some embodiments, the beta radiation is Strontium-90 (Sr-90), Phosphorus-32 (P-32), Ruthenium 106 (Ru-106), Yttrium 90 (Y-90), or a combination thereof.

Examples of the present invention include but are not limited to a radioisotope that emits beta radiation for use in a method of treating both glaucoma and cataracts, a radioisotope that emits beta radiation for use for use in preventing or reducing scar formation in a drainage bleb or drainage channel in an eye being treated or having been treated with glaucoma surgery and cataract surgery, a radioisotope that emits beta radiation for use for use in a method for reducing intraocular pressure (IOP) in an eye being treated or having been treated with glaucoma surgery and cataract surgery, a composition comprising a source of beta radiation for use in a method for achieving a healthy intraocular pressure (IOP) in a human eye being treated or having been treated for glaucoma and cataracts, etc. The radioisotope or composition may be administered to the eye such that beta radiation from the source of beta radiation is applied to a target area of the eye, wherein the target area is associated with the drainage bleb, a drainage channel, or a glaucoma drainage implant.

Examples of methods of the present invention include but are not limited to methods of treating both glaucoma and cataracts, methods of preventing or reducing scar formation in a drainage bleb or drainage channel in an eye being treated or having been treated with glaucoma surgery and cataract surgery, methods for reducing intraocular pressure (IOP) in an eye being treated or having been treated with glaucoma surgery and cataract surgery, methods for achieving a healthy intraocular pressure (IOP) in a human eye being treated or having been treated for glaucoma and cataracts, etc.

The glaucoma surgery allows aqueous humor to drain into a bleb in a subconjunctival space or space between a conjunctiva and Tenon's capsule. In certain embodiments, the glaucoma surgery is Minimally Invasive Glaucoma Surgery (MIGS).

In certain embodiments, the methods herein are effective for one or a combination of; maintaining a functioning drainage bleb; inhibiting or reducing fibrogenesis and inflammation in the bleb, around the drainage implant, or around the drainage channel; and reducing conjunctival inflammation. In certain embodiments, the methods herein are effective for achieving a healthy IOP. In certain embodiments, the methods herein are effective for maintaining a healthy IOP. In certain embodiments, the methods herein are effective for lowering IOP and maintaining said IOP.

Inhibiting or reducing fibrogenesis and inflammation in the bleb may be measured according to a predetermined bleb grading scale. In certain embodimetns, the predetermined bleb grading scale is Moorfields bleb grading scale (MBGS). In certain embodimetns, the predetermined bleb grading scale is Indiana Bleb Appearance Grading Scale (IBAGS).

In certain embodiments, methods herein comprise performing a glaucoma drainage surgery in an eye and performing cataract surgery on the eye. In certain embodiments, the methods herein are performed after glaucoma drainage surgery and/or cataract surgery has been performed.

The methods herein feature applying a therapeutic dose of the beta radiation from a radioisotope or composition or source to a target area of the eye. The target area may be associated with the bleb. In certain embodiments, the target area is associated with a glaucoma drainage implant, In certain embodiments, the target area is associated with a drainage channel.

In certain embodiments, the beta radiation is applied to the target after performing the glaucoma drainage surgery. In certain embodiments, the beta radiation is applied to the target before performing the glaucoma drainage surgery. In certain embodiments, the beta radiation is applied to the target while performing the glaucoma drainage surgery. In certain embodiments, the beta radiation is applied to the target before and after performing the glaucoma drainage surgery.

In certain embodiments, the methods herein further comprise administering a drug to the target area. Non-limiting examples of drugs include mitomycin C, 5 fluorouracil, an anti-VEGF composition, etc.

In certain embodiments, intraocular pressure (IOP) is reduced to 12 mmHg or less. In certain embodiments, IOP is reduced to 10 mmHg or less. In certain embodiments, IOP is reduced to from 5 to 10 mmHg. In certain embodiments, IOP is reduced to from 5 to 12 mmHg. In certain embodiments, IOP is reduced to from 8 to 10 rnmHg. T In certain embodiments, IOP is reduced to from 8 to 12 mmHg.

In some embodiments, the method is effective for reducing IOP by 10% or more 6 months after treatment. In some embodiments, the method is effective for reducing IOP by 20% or more 6 months after treatment, In some embodiments, the method is effective for reducing IOP by 30% or more 6 months after treatment. In some embodiments, the method is effective for reducing IOP by 40% or more 6 months after treatment. In some embodiments, the method is effective for reducing IOP by 50% or more 6 months after treatment.

In some embodiments, the method is effective for reducing IOP by 10% or more 12 months after treatment. In some embodiments, the method is effective for reducing IOP by 20% or more 12 months after treatment. In some embodiments, the method is effective for reducing IOP by 30% or more 12 months after treatment. In some embodiments, the method is effective for reducing IOP by 40% or more 12 months after treatment. In some embodiments, the method is effective for reducing IOP by 50% or more 12 months after treatment.

In some embodiments, the method is effective for reducing IOP by 10% or more 24 months after treatment. In some embodiments, the method is effective for reducing IOP by 20% or more 24 months after treatment. In some embodiments, the method is effective for reducing IOP by 30% or more 24 months after treatment. In some embodiments, the method is effective for reducing IOP by 40% or more 24 months after treatment. In some embodiments, the method is effective for reducing IOP by 50% or more 24 months after treatment.

In some embodiments, the method is effective for reducing IOP by 10% or more 36 months after treatment. In some embodiments, the method is effective for reducing IOP by 20% or more 36 months after treatment. In some embodiments, the method is effective for reducing IOP by 30% or more 36 months after treatment. In some embodiments, the method is effective for reducing IOP by 40% or more 36 months after treatment. In some embodiments, the method is effective for reducing IOP by 50% or more 36 months after treatment.

In some embodiments, the method is effective for reduction of IOP and subsequent stabilization of IOP. In some embodiments, stabilization of IOP is wherein the IOP does not increase by more than 10% at 3 months after treatment. In some embodiments, stabilization of IOP is wherein the IOP does not increase by more than 10% at 6 months after treatment. In some embodiments, stabilization of IOP is wherein the IOP does not increase by more than 10% at 12 months after treatment. In some embodiments, stabilization of IOP is wherein the IOP does not increase by more than 10% at 24 months after treatment. In some embodiments, stabilization of IOP is wherein the IOP does not increase by more than 10% at 36 months after treatment.

In some embodiments, stabilization of IOP is wherein the IOP does not increase by more than 20% at 3 months after treatment. In some embodiments, stabilization of IOP is wherein the IOP does not increase by more than 20% at 6 months after treatment. In some embodiments, stabilization of IOP is wherein the IOP does not increase by more than 20% at 12 months after treatment. In some embodiments, stabilization of IOP is wherein the IOP does not increase by more than 20% at 24 months after treatment. In some embodiments, stabilization of IOP is wherein the IOP does not increase by more than 20% at 36 months after treatment.

In some embodiments, stabilization of IOP is wherein the IOP does not increase by more than 25% at 24 months after treatment. In some embodiments, stabilization of IOP is wherein the IOP does not increase by more than 25% at 36 months after treatment.

In some embodiments, the systems and devices of the present invention may be used for methods associated with needling procedures, e.g., procedures to the bleb to free or remove scar tissue and/or cystic structures in and/or around the bleb and/or surgery site that may later arise from wound healing or scarring or inflammatory responses to the glaucoma surgery. Needling procedures may affect surgical site morphology, restore the function of the surgery and/or lower the IOP.

### EXAMPLE 1

### Surgical Procedure for Beta Radiation Application

The present invention provides an example of a procedure for the application of beta radiation to the eye. The present invention is in no way limited to the specific steps, methods, devices, systems, and compositions described herein.

### Preparation and Assembly

The device assembly procedure may be done behind a plexiglass beta shield (for example, the Large Dual Angle Beta Radiation Shield, Universal Medical Inc.). The medical technician or medical physicist or other user opens the Radioisotope Brachytherapy Source (RBS) storage container. The RBS is removed from its container using appropriate handling techniques (for example, long forceps). The RBS is placed on a clean field.

The Brachytherapy Applicator may be a single-use sterile-packed device. Its packaging may be checked by examining for damage or breach of the sterile barrier. If finding none, the Brachytherapy Applicator package is opened, and the applicator assembly placed on a sterile field.

The Brachytherapy Applicator comprises a handle and an RBS cap. Using aseptic technique and remote handling techniques, the RBS is loaded into the Brachytherapy Applicator, e.g., the RBS may be inserted into the cap and the handle is subsequently connected to the cap, securing the RBS. Care is taken to avoid contamination.

The radiation output may be confirmed consistent with standards of quality assurance in radiation therapy (for example see: Palmer, Antony L., Andrew Nisbet, and David Bradley. “Verification of high dose rate brachytherapy dose distributions with EBT3 Gafchromic film quality control techniques.” Physics in medicine and biology 58.3 (2013): 497). In one method of quality assurance, the applicator is applied to radiographic film in sterile overwrap for a specified dwell time (for example Gafchromic® film, Ashland Inc.), The overwrap is removed. The medical physicist checks the area of application for evidence of film exposure.

The device may be placed into a sterile plexiglass beta transport box (for example the IBI Beta-Gard Acrylic Storage Container—Large, Universal Medical Inc.) and the box placed on the operative Mayo stand.

Previously the decayed activity of the RBS has been calculated to determine the contemporary dose per unit time (for example, cGy/second). The decay calculation methodology is known to those skilled in medical physics and is also described in the NRC Information Notice 96-66: United States Nuclear Regulatory Commission, Office of Nuclear Material Safety and Safeguards, Washington D.C. 20555, Dec. 13, 1996. The dwell time for the total prescribed dose is then calculated. As an example, the prescription dose is 1,000 cGy to a center point of 0.19 mm depth from the conjunctival surface. As an example, the decayed activity of the RBS is 30 cGy/second at a water equivalent depth of 0.19 mm. In this example, the dwell time is calculated to be about 33 seconds, providing a 990 cGy dose.

### Surgical Application

The beta therapy may be applied following completion of a glaucoma surgery. (Note the present invention is not limited to applying beta radiation after glaucoma surgery.) The eye is rotated to a downward gaze position by the use of a probe placed against the sclera providing traction (for example the distal end of a Vera Hook placed against the eye). This allows better visual and surgical access to the superior conjunctiva.

The ophthalmic surgeon obtains the Brachytherapy Applicator device, e.g., from the transport box. The tip (e.g., distal end, active end) of the applicator is placed over the conjunctiva in a position just superior to the limbus. The diameter of the applicator encompasses the appropriate surface area of the target, e.g., bleb. The Brachytherapy Applicator is pressed to the surface of the eye. In some embodiments, the Brachytherapy Applicator is pressed to the surface of the eye such that all or substantially all of the edema fluid is pushed away. The Applicator is held in place for the specified dwell time. In some embodiments, the dwell time has been programmed into a count-down dock. Following the specified dwell time, the Brachytherapy Applicator is removed from the operative field.

At the conclusion of surgery, antibiotic ointment is applied to the eye and the eye patched.

In certain embodiments, following the surgery, the Brachytherapy Applicator is disassembled behind the acrylic beta shield. The Radioisotope Brachytherapy Source is returned to its storage container. The disposable portions of the device are discarded in a manner consistent with appropriate disposal of biological waste (for example “red bag” waste).

### EXAMPLE 2

### Surgical Procedure for Phacoemulsification and Trabeculectomy

The present invention provides an example of a procedure for the extraction of cataracts followed by trabeculectomy. The present invention is in no way limited to the specific steps, methods, devices, systems, and compositions described herein.

Removal of the natural lens from the eye: An opening is created in the dear front window of the eye (cornea). A circular opening is created in the front aspect of the bag (capsule) that contains the natural lens of the eye. The natural lens is removed from within the capsule using ultrasound to break it up (phacoemulsification) and suction.

Introduction of an intraocular lens: A clear artificial lens is folded and introduced into the capsule through the same corneal opening. It unfolds in the capsule.

Creation of a trapdoor in the sclera. The thin layer (conjunctiva) covering the white aspect (sclera) of the front of the eye is reflected back by cutting its attachment at the front. A trap door is created in the sclera hinged at the front (by the cornea).

An opening into the front chamber of the eye at the base/hinge of the trapdoor: A hole (trabeculectomy) is created through the sclera into the front chamber of the eye, a hole is also made in the iris to stop the iris floating up and blocking the scleral hole. The trap door is closed with special stitches that can be removed to open it out if needed after the operation.

Closure of the conjunctiva over the trapdoor: The conjunctiva is reattached at the edge of the cornea to cover the trap door and its opening. The red line shows the flow of fluid (aqueous humour) from the front chamber of the eye out through the trapdoor to a space under the conjunctiva creating a bleb.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

Any reference numbers recited herein, including the claims below, are solely for ease of examination of this patent application, and are exemplary, and are not intended in any way to limit the scope of the claims to the particular features having the corresponding reference numbers in the drawings.

1.-115. (canceled)

116. A method of maintaining a functioning drainage bleb., reducing scar formation in a drainage bleb, or reducing conjunctival inflammation in an eye being treated or having been treated with (i) glaucoma drainage surgery wherein an implant is implanted trans-sclerally to form a bleb in a subconjunctival space or between the conjunctiva and Tenon's capsule and aqueous humor can drain into the drainage bleb, and (ii) cataract surgery, said method comprising applying a therapeutic dose of beta radiation from a radioisotope to a target area of the eye, wherein the target area is associated with the bleb, the implant, or both the bleb and implant; wherein the beta radiation causes cell cycle arrest in fibroblasts on the Tenon's capsule to inhibit or reduce the fibrotic process and conjunctival inflammation, reduce scar formation, or maintain a functioning drainage bleb.

117. (canceled)

118. The method of claim 116, wherein the glaucoma surgery is Minimally Invasive Glaucoma Surgery (MIGS) and the implant is a MIGS implant.

119. (canceled)

120. The method of claim 116, wherein the radioisotope comprises Strontium-90 (Sr-90), Phosphorus-32 (P-32), Ruthenium 106 (Ru-106), Yttrium 90 (Y-90), or a combination thereof.

121. (canceled)

122. The method of claim 116, wherein the therapeutic dose is from 450-1050 cGy.

123.-125. (canceled)

126. The method of claim 116, wherein the beta radiation is applied to the target using an applicator.

127.-136. (canceled)

137. The method of claim 116, wherein the method is effective for reducing IOP.

138. The method of claim 137, wherein the method is effective for reducing IOP and subsequent stabilization of said IOP.

139.-143. (canceled)

144. The method of claim 138, wherein stabilization of IOP is wherein the IOP does not increase by more than 20% at 3 months after treatment.

145. The method of claim 138, wherein stabilization of IOP is wherein the IOP does not increase by more than 20% at 6 months after treatment.

146.-150. (canceled)

151. A method of treating glaucoma and cataracts, said method comprising:
a. performing a glaucoma drainage surgery in an eye, wherein an implant is implanted trans-sclerally to form a bleb in a subconjunctival space or between the conjunctiva and Tenon's capsule and aqueous humor can drain into the drainage bleb;
b. performing cataract surgery on the eye; and
c. applying a therapeutic dose of the beta radiation from a radioisotope to a target area of the eye, wherein the target area is associated with the bleb, the implant, or both the bleb and implant;
wherein the method is effective for one or a combination of: reducing intraocular pressure (IOP), maintaining a functioning drainage bleb; inhibiting or reducing fibrogenesis and inflammation in the bleb, around the drainage implant, or around the drainage channel; and reducing conjunctival inflammation in the eye.

152. The method of claim 151, wherein the glaucoma drainage surgery is Minimally Invasive Glaucoma Surgery (MIGS) and the implant is a MIGS implant.

153. (canceled)

154. The method of claim 151, wherein the radioisotope comprises Strontium-90 (Sr-90), Phosphorus-32 (P-32), Ruthenium 106 (Ru-106), Yttrium 90 (Y-90), or a combination thereof.

155. (canceled)

156. The method of claim 151, wherein the therapeutic dose is from 450-1050 cGy.

157.-159. (canceled)

160. The method of claim 151, wherein the beta radiation is applied to the target after performing the glaucoma drainage surgery.

161. The method of claim 151, wherein beta radiation is applied to the target before performing the glaucoma drainage surgery.

162. The method of claim 151, wherein a beta radiation is applied to the target while performing the glaucoma drainage surgery.

163. The method of claim 151, wherein beta radiation is applied to the target before and after performing the glaucoma drainage surgery.

164.-170. (canceled)

171. The method of claim 151, wherein the method is effective for reducing IOP and subsequent stabilization of said IOP.

172.-184. (canceled)

185. The method of claim 151, wherein inhibiting or reducing fibrogenesis and inflammation in the bleb is measured according to a predetermined bleb grading scale.

186. The method according to claim 185, wherein the predetermined bleb grading scale is Moorfields bleb grading scale (MBGS) or Indiana Bleb Appearance Grading Scale (IBAGS).

187. (canceled)
